(19) United States
(12) Patent Application Publication
(10) Pub. No.: US 2009/0246256 A1
(43) Pub. Date: Oct. 1, 2009

(54) Compositions and Methods for Transmucosal Delivery of Lofexidine

(76) Inventor: Abeer M. Al-Ghananeem, Lexington, KY (US)

Correspondence Address: FROST BROWN TODD, LLC, 2200 PNC CENTER, 201 E. FIFTH STREET, CINCINNATI, OH, 45202, US

(21) Appl. No.: 12/410,114

(22) Filed: Mar. 24, 2009

Related U.S. Application Data
(60) Provisional application No. 61/039,977, filed on Mar. 27, 2008.

(51) Int. Cl. A61F 13/02; A61K 9/68; A61K 9/127; A61K 9/20; A61K 9/10; A61K 9/14; A61K 31/415

(52) U.S. Cl. 424/440; 424/448; 424/450; 424/464; 424/484; 424/490; 514/401

(57) ABSTRACT

The present invention provides for compositions and methods for accelerating the rate of delivery of lofexidine to the systemic circulation by transmucosal administration through the nasal, sublingual, or buccal routes to provide rapid response in the treatment of opiate addicts, migraine, neuropathic pain, and other therapeutic indications related to lofexidine, to a patient in need of such treatment. Compositions of lofexidine formulated for transmucosal delivery are provided. Also provided are methods for the treatment of opiate addicts, migraine, neuropathic pain, and other therapeutic indications related to lofexidine. The methods utilize lofexidine compositions formulated for transmucosal delivery through nasal, sublingual, or buccal routes of administration in an amount effective for the treatment of the drug indications.

### CROSS-REFERENCE TO RELATED APPLICATIONS

This application claims priority to U.S. Provisional Patent Application Ser. No. 61/039,977, which was filed on Mar. 27, 2008, the entirety of which is incorporated herein by reference for all purposes.

### FIELD OF THE INVENTION

The present invention relates to methods for delivering active agents comprising lofexidine or a pharmaceutically acceptable free base, salt, ester, amide or prodrug thereof, through transmucosal administration. More specifically, the invention features compositions and methods for the transmucosal administration of lofexidine through nasal, sublingual, and buccal routes of administration for the treatment of opiate addicts, migraine, neuropathic pain, and other therapeutic indications related to lofexidine. The dosage forms are those suitable for transmucosal drug delivery, such as spray, drops, gels, tablets, troches, lozenges, chewing gum, patches, etc.

### BACKGROUND OF THE INVENTION

Lofexidine is an α2-adrenergic receptor agonist analogue of clonidine that acts centrally to suppress opiate withdrawal symptoms. The drug has been available for use as a non-opioid medication for opioid detoxification in the United Kingdom under the label BritLofex since 1992. Lofexidine was reported to be metabolized after oral delivery more extensively than the related anti-hypertensive agent, clonidine. The principal metabolite of lofexidine was reported to be 2,6-dichlorophenol, which was apparently excreted in urine as two O-glucuronic acid conjugates.

Despite the overall popularity for the oral drug delivery route, transmucosal delivery routes are attractive and particularly advantageous due to their non-invasive nature. Transmucosal delivery routes such as nasal, sublingual and buccal are usually simple and can be administered by the caregiver or the patient with minimal discomfort.

Furthermore, transmucosal drug delivery will minimize the peripheral side effects that might be associated with oral lofexidine administration, and will deliver lofexidine effectively to the blood stream for the detoxification of addicts by-passing the first pass metabolism by the liver.

Transmucosal delivery will afford a rapid absorption, high bioavailability; therefore, lower doses and fewer side effects. In addition, the avoidance of first-pass metabolism by the liver and metabolism by the gastrointestinal tract will diminish the possible side effects associated with lofexidine metabolites.

Accordingly, it is one of the purposes of this invention, among others, to provide safe and reliable intranasal, sublingual or buccal delivery systems for lofexidine that ensure delivery of therapeutic amounts of the drug into the bloodstream which is fast acting, easily administered and causes no substantial adverse side effects.

### BRIEF SUMMARY OF THE INVENTION

The present invention relates to compositions comprising lofexidine for transmucosal delivery. The compositions are suitable for sublingual, nasal, and buccal administration use, and provide for absorption of the drug across the oral and nasal mucosa.

The invention is also directed to methods of treatment comprising administering lofexidine by transmucosal delivery. The inventive methods may improve bioavailability relative to oral dosage forms, especially in those patients with abnormally slow gastric emptying. Such methods can involve administration of the novel lofexidine containing compositions described herein. The methods may provide treatment for a variety of conditions amenable to amelioration by lofexidine administration, without the occurrence of possible side effects associated with oral ingestion.

The present invention provides a pharmaceutical composition comprising lofexidine or a pharmaceutically acceptable salt thereof and a pharmaceutically acceptable carrier, wherein the lofexidine or pharmaceutically acceptable salt thereof is provided in a form suitable for transmucosal delivery through nasal, sublingual, or buccal administration.

These and other embodiments of the invention are described herein below or are evident to persons of ordinary skill in the art based on the following disclosures.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

### BRIEF DESCRIPTION OF THE DRAWINGS

This invention, as defined in the claims, can be better understood with reference to the following drawings:

FIG. 1 illustrates the mean plasma lofexidine levels following sublingual and intravenous administration at 1 mg/kg single dose in rabbits (n=3). All values show the mean±STDV.

In the following description of the illustrated embodiments, references are made to the accompanying drawings, which form a part hereof, and in which is shown by way of illustration various embodiments in which the invention may be practiced. It is to be understood that other embodiments may be utilized and structural and functional changes may be made without departing from the scope of the present invention.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for all purposes including for describing and disclosing the cell lines, vectors, and methodologies which are reported in the publications which might be used in connection with the invention. Nothing herein is to be construed as an admission that the invention is not entitled to antedate such disclosure by virtue of prior invention.

In order to provide a clear and consistent understanding of the specification and claims, including the scope to be given such terms, the following definitions are provided:

The term “administration” of the pharmaceutically active compounds and the pharmaceutical compositions defined herein includes transmucosal application. Nasal, sublingual and buccal administration are particularly preferred in the present invention.

“Ameliorate” or “amelioration” means a lessening of the detrimental effect or severity of the disease in the subject receiving therapy, the severity of the response being determined by means that are well known in the art.

By “compatible” herein is meant that the components of the compositions which comprise the present invention are capable of being comingled without interacting in a manner which would substantially decrease the efficacy of the pharmaceutically active compound under ordinary use conditions.

The terms “effective amount” or “pharmaceutically effective amount” refer to a nontoxic but sufficient amount of the agent to provide the desired biological result. That result can be reduction and/or alleviation of the signs, symptoms, or causes of a disease, such as neural diseases and malignant hyperthermia, or any other desired alteration of a biological system. Such amounts are described below. An appropriate “effective” amount in any individual case may be determined by one of ordinary skill in the art using routine experimentation.

As used herein, the term “excipient” means the substances used to formulate active pharmaceutical ingredients (API) into pharmaceutical formulations; in a preferred embodiment, an excipient does not lower or interfere with the primary therapeutic effect of the API. Preferably, an excipient is therapeutically inert. The term “excipient” encompasses carriers, diluents, vehicles, solubilizers, stabilizers, bulking agents, acidic or basic pH-adjusting agents and binders. Excipients can also be those substances present in a pharmaceutical formulation as an indirect or unintended result of the manufacturing process. Preferably, excipients are approved for or considered to be safe for human and animal administration, i.e., GRAS substances (generally regarded as safe). GRAS substances are listed by the Food and Drug administration in the Code of Federal Regulations (CFR) at 21 CFR 182 and 21 CFR 184, incorporated herein by reference.

As used herein, the terms “formulate” refers to the preparation of a drug, e.g., lofexidine, in a form suitable for administration to a mammalian patient, preferably a human. Thus, “formulation” can include the addition of pharmaceutically acceptable excipients, diluents, or carriers and pH adjusting agents.

The term “permeation enhancer” or “penetration enhancer” as used herein refers to an agent that improves the rate of transport of a pharmacologically active agent (e.g., lofexidine) across the mucosal surface. Typically a penetration enhancer increases the permeability of mucosal tissue to a pharmacologically active agent. Penetration enhancers, for example, increase the rate at which the pharmacologically active agent permeates through membranes and enters the bloodstream. Enhanced permeation effected through the use of penetration enhancers can be observed, for example, by measuring the flux of the pharmacologically active agent across animal or human membranes as described in the Examples herein below. An “effective” amount of a permeation enhancer as used herein means an amount that will provide a desired increase in mucosal membranes permeability to provide, for example, the desired depth of penetration of a selected compound, rate of administration of the compound, and amount of compound delivered.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

As used herein, a “pharmaceutically acceptable carrier” is a material that is nontoxic and generally inert and does not affect the functionality of the active ingredients adversely. Examples of pharmaceutically acceptable carriers are well known and they are sometimes referred to as dilutents, vehicles or excipients. The carriers may be organic or inorganic in nature. In addition, the formulation may contain additives such as flavoring agents, coloring agents, thickening or gelling agents, emulsifiers, wetting agents, buffers, stabilizers, and preservatives such as antioxidants.

The term “pharmaceutical composition” as used herein shall mean a composition that is made under conditions such that it is suitable for administration to humans, e.g., it is made under GMP conditions and contains pharmaceutically acceptable excipients, e.g., without limitation, stabilizers, pH adjusting agents, bulking agents, buffers, carriers, diluents, vehicles, solubilizers, and binders.

The liquid carrier or vehicle can be a solvent or liquid dispersion medium comprising, for example, water, ethanol, a polyol such as glycerol, propylene glycol, or liquid polyethylene glycols and the like, vegetable oils, nontoxic glyceryl esters, and suitable mixtures thereof. The prevention of the growth of microorganisms can be accomplished by various antibacterial and antifungal agents, for example, parabens, chlorobutanol, phenol, sorbic acid, thimerosal, and the like.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, such as by suppressing or relieving the disease, i.e., causing regression of the disease or its clinical symptoms.

General

The present invention provides for compositions and methods for the delivery of lofexidine, or pharmaceutically acceptable salt thereof, to a patient in need of such treatment, comprising the transmucosal administration of lofexidine. More specifically, the invention features dosage forms compositions and methods for the transmucosal delivery of lofexidine through nasal, sublingual, and buccal routes of administration for the treatment of opiate addicts, migraine, neuropathic pain, and other therapeutic indications related to lofexidine. The dosage forms are those suitable for transmucosal drug delivery, such as spray, drops, gels, tablets, troches, lozenges, chewing gum, patches, etc. The transmucosal delivery through nasal, sublingual or buccal dosage form administration offers significant clinical advantages over the prior art. More specifically, the invention sought to provide a rapid, reliable, safe, effective and convenient treatment for therapeutic indications related to lofexidine such as a treatment to relieve symptoms in patients undergoing opiate detoxification, to decrease stress-induced reinstatement of seeking addictive materials, and to treat pain management in general such as migraine and neuropathic pain, which comprises the administration of lofexidine through nasal, sublingual, and/or buccal administration, thus providing rapid response compared to prior art methods of administering lofexidine, while avoiding the side-effects associated with oral dosage forms.

The invention is directed to a pharmaceutical composition comprising from about 0.05 mg to about 5 mg of lofexidine. In some embodiments, the pharmaceutical composition comprises from about 0.1 mg to about 1 mg of lofexidine.

The invention is directed to pharmaceutical compositions comprising from about 0.05 mg to about 5 mg of lofexidine wherein the composition further comprises a permeation enhancer.

In one aspect, the present invention relates to a composition for pharmaceutical drug delivery. The composition may be formulated to be suitable for systemic application, for example, transmucosal applications. The composition typically comprises a therapeutically effective amount of lofexidine or a pharmaceutically acceptable salt or derivative thereof.

In another embodiment, the composition comprises a gelling or thickening agent(s). Exemplary gelling agents include, but are not limited to, carbomer, carboxyethylene or polyacrylic acid such as carbomer 980 or 940 NF, 981 or 941 NF, 1382 or 1342 NF, 5984 or 934 NF, ETD 2020, 2050, 934P NF, 971P NF, 974P NF, polycarbophils such as NOVEON AA-1, NOVEON CA1/CA2, carbomer copopolymers such as PEMULEN TR1 NF or PEMULEN TR2 NF, carbomer interpolymers such as CARBOPOL ETD 2020 NF, CARBOPOL ETD 2050 NF, CARBOPOL ULTRA EZ 10, etc. . . . ; cellulose derivatives such as ethylcellulose, hydroxypropylmethylcellulose (HPMC), ethyl-hydroxyethylcellulose (EHEC), carboxymethylcellulose (CMC), hydroxypropylcellulose (HPC), hydroxyethylcellulose (HEC), etc. . . . ; natural gums such as arabic, xanthan, guar gums, alginates, etc. . . . ; polyvinylpyrrolidone derivatives; polyoxyethylene polyoxypropylene copolymers, etc; others like chitosan, polyvinyl alcohols, pectins, veegum grades, and the like. Other suitable gelling agents to apply the present invention include, but are not limited to, carbomers. Alternatively, other gelling agents or viscosant known by those skilled in the art may also be used. The gelling agent or thickener is present from about 0.2 to about 30% w/w depending on the type of polymer, as known by one skilled in the art. A preferred concentration range of the gelling agent(s), for example, hydroxypropyl cellulose or carbomer, is a concentration of between about 0.5 and about 5 weight percent, more preferred is a concentration of between about 1 and about 3 weight percent.

In another embodiment, the composition comprises a permeation enhancer (penetration enhancer). Permeation enhancers include, but are not limited to, sulfoxides such as dimethylsulfoxide and decylmethylsulfoxide; surfactants such as sodium laurate, sodium lauryl sulfate, cetyltrimethylammonium bromide, benzalkonium chloride, poloxamer (231, 182, 184), tween (20, 40, 60, 80) and lecithin; the 1-substituted azacycloheptan-2-ones, particularly 1-n-dodecylcyclazacycloheptan-2-one; fatty alcohols such as lauryl alcohol, myristyl alcohol, oleyl alcohol and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate, amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine, terpenes; alkanones, and organic acids, particularly salicylic acid and salicylates, citric acid and succinic acid. As noted earlier herein, “Percutaneous Penetration Enhancers”, eds. Smith et al. (CRC Press, 1995), which is incorporated herein by reference thereto, provides an excellent overview of the field and further information concerning possible secondary enhancers for use in conjunction with the present invention. More permeation enhancer(s) suitable to be used with the present invention may be known by those skilled in the art. The permeation enhancer is present from about 0.1 to about 30% w/w depending on the type of compound. Preferred permeation enhancers are fatty alcohols and fatty acids. More preferred permeation enhancers are fatty alcohols. Preferably, the fatty alcohols have the formula the CH₃(CH₂)ₙ(CH)ₘCH₂OH wherein n ranges from (8-m) to (16-m) and m=0-2. A preferred concentration range of the penetration enhancer(s) is, depending on the type of permeation enhancer, a concentration of between about 0.1 and about 10 weight percent, as known by one skilled in the art. In one preferred embodiment, the penetration enhancer comprises myristyl alcohol in a concentration of between about 0.1 and about 2 weight percent.

In some embodiments, the permeation enhancer is chosen from: a bile salt, sodium dodecyl sulfate, dimethyl sulfoxide, sodium lauryl sulfate, a derivative of a saturated or unsaturated fatty acid, a surfactant, a bile salt analog, and a derivative of a bile salt. In some embodiments the permeation enhancer is a synthetic permeation enhancer.

In another embodiment, the composition comprises antioxidant(s), for example, tocopherol and derivatives, ascorbic acid and derivatives, butylated hydroxyanisole, butylated hydroxytoluene, fumaric acid, malic acid, propyl gallate, sodium metabisulfite and derivatives, is a concentration of about 0.01 to about 5 weight percent; more preferred is a concentration of about 0.1 to about 0.5 weight percent, depending on the type of antioxidant used, as known by the one skilled in the art.

In another embodiment, the composition comprises buffering agent(s), for example, carbonate buffers, citrate buffers, phosphate buffers, acetate buffers, hydrochloric acid, lactic acid, tartaric acid, inorganic and organic bases, is a concentration of about 1 to about 10 weight percent, more preferred is a concentration of about 2 to about 5 weight percent, depending on the type of buffering agent(s) used, as known by the one skilled in the art. The preferred concentration range of said buffering agents are those enabling design of compositions having a pH close to the physiologic pH of the mucosal membranes, between about pH 2.0 and about pH 10.0, preferably between about pH 3.0 and pH 7.0. Concentrations of the buffering agent(s) may vary, however, as known by the one skilled in the art. The buffering agent may replace up to 100% of the water amount within the composition.

The transmucosal pharmaceutical formulation of the present invention may also further include preservatives such as benzalkonium chloride and derivatives, benzoic acid, benzyl alcohol and derivatives, bronopol, parabens, cetrimide, chlorhexidine, cresol and derivatives, imidurea, phenol, phenoxyethanol, phenylethyl alcohol, phenylmercuric salts, thimerosal, sorbic acid and derivatives. The preservative is present from about 0.01 to about 10% w/w depending on the type of compound used, as known by the one skilled in the art.

The transmucosal pharmaceutical formulation of the present invention may also further include humectants, sequestering agents, moisturizers, surfactants, emollients, colorants, fragrances, flavors, or any combination thereof.

In some embodiments, the transmucosal dosage form is a liquid formulation, comprising: lofexidine or pharmaceutically acceptable salt thereof, aqueous solvent; and a polar organic solvent, wherein the polar organic solvent is present in an amount sufficient to enhance the solubility of the lofexidine free base or salt thereof in the water.

In one embodiment, a gel formulation of the present invention comprises a therapeutically effective amount of a lofexidine, or a pharmaceutically acceptable salt or derivative thereof, of between about 0.01 to about 5 weight percent. The primary vehicle may comprise between about 10 to about 60 weight percent of water, between about 30 to about 70 weight percent ethanol, between about 15 and about 60 weight percent of a 10:1 to 1:10 (weight to weight) mixture of diethylene glycol mono ethyl ether and propylene glycol, and between about 0.1 and about 2 weight percent of lauryl alcohol, myristyl alcohol, oleyl alcohol, lauric acid, myristic acid, or oleic acid. The primary vehicle may be gellified with between about 0.5 and about 5 weight percent of hydroxypropylcellulose. The apparent pH of the gel is between about pH 2.0 and about pH 10.0, or preferably between about pH 3.0 and pH 7.0.

In another embodiment, the lofexidine or pharmaceutically acceptable salt thereof, is present at a dose of 0.05-5 mg of lofexidine. Preferably, the polar organic solvent is an alcohol. The alcohol may include, but is not limited to, ethanol, propylene glycol, glycerol, polyethylene glycol and mixtures thereof. More preferably, the alcohol is ethanol. Preferably, the polar organic solvent is present in an amount of 0.5-50% w/w.

In addition, the transmucosal delivery system of the pharmaceutical composition can include a buffer to maintain the pH of the formulation and a pharmaceutically acceptable thickening agent. The pharmaceutical composition can further include one or more pharmaceutical excipients and even further include a pharmaceutically acceptable preservative.

The buffer of the transmucosal delivery system can be selected from the group including acetate, citrate, prolamine, carbonate and phosphate buffers.

The thickening agent of the transmucosal delivery system can be selected from the group including methyl cellulose, xanthan gum, carboxymethyl cellulose, hydroxypropyl cellulose, carbomer, polyvinyl alcohol, alginates, acacia, chitosans and combinations thereof.

The formulation may further comprise a sweetener suitable for sublingual and buccal delivery systems. The sweetener may be, but is not limited to, mannitol, saccharin or saccharin sodium. The formulation may further comprise a flavoring agent. Preferably, the flavoring agent is menthol. The formulation may further comprise a thickening agent. The thickening agent may be, but is not limited to, methyl cellulose, xanthan gum, carboxymethyl cellulose, hydroxypropyl cellulose, carbomer, polyvinyl alcohol, alginates, acacia, chitosans and combinations thereof.

The formulation may further comprise a humectant suitable for nasal delivery system. The humectant may be, but not limited to, sorbitol, glycerol, mineral oil, vegetable oil and combinations thereof.

In some embodiments, the transmucosal carrier of the transmucosal dosage unit is preferably an aqueous solution. Further, the aqueous solution can be selected from the group including aqueous gels, aqueous suspensions, aqueous liposomal dispersions, aqueous emulsions, aqueous microemulsions, aqueous nanoparticles and combinations thereof.

Alternatively, the carrier of the transmucosal dosage unit is a nonaqueous solution. The non-aqueous solution can be selected from a group including non-aqueous gels, non-aqueous suspensions, non-aqueous liposomal dispersions, nonaqueous emulsions, non-aqueous microemulsions, non-aqueous nanoparticles and combinations thereof.

The carrier of the transmucosal dosage unit can also be a combination of an aqueous solution and a non-aqueous solution. The formulation may be partially pressurized. Alternatively, the carrier of the transmucosal dosage unit is a powder formulation.

The powder formulation can be selected from, but not limited to, a simple powder mixtures, powder microspheres, coated powder microspheres, liposomal dispersions and combinations thereof. Preferably, the powder formulation is simple powder mixture.

In some embodiments the oral transmucosal dosage form is chosen from: a chewing gum, a patch, a lozenge, a tablet, a troche, a pastille, a sachet, and a rapid disintegrating tablet.

The formulations of the present invention may be provided in a unit dose container(s). Such containers typically comprise inner and outer surfaces, wherein the formulation of the present invention is contained by the inner surface of the container. In selected embodiments, the container is a packet or a vial, and the inner surface of the container may further comprise a liner. For example, in one embodiment, the container is a flexible, foil packet and the liner is a polyethylene liner. Alternatively, or in addition, the formulations of the present invention may be provided in a multiple dose container(s). Such multiple dose containers typically comprise inner and outer surfaces, wherein the gel for pharmaceutical drug delivery is contained by the inner surface of the container. Multiple dose containers may, for example, dispenses fixed or variable metered doses. Multiple dose containers may, for example, be a stored-energy metered dose pump or a manual metered dose pump.

In another aspect the present invention comprises a composition for pharmaceutical drug delivery, comprising a therapeutically effective amount of lofexidine, or a pharmaceutically acceptable salt or derivative thereof, in a hydroalcoholic vehicle comprising water, a short chain alcohol, a monoalkyl ether of diethylene glycol, a pharmaceutically acceptable glycol, and an optional fatty permeation enhancer. In such compositions the pH of the composition is typically between about pH 2.0 and about pH 9.0. These compositions for pharmaceutical delivery may include further components as described herein, for example, the hydroalcoholic vehicle may further comprise a permeation enhancer. Such compositions may be formulated in a variety of ways including wherein the hydroalcoholic vehicle is gellified. These compositions may be used, for example, for transmucosal applications including application to nasal, sublingual and buccal tissues.

In yet another aspect the present invention comprises a composition for pharmaceutical drug delivery, comprising a therapeutically effective amount of lofexidine, or a pharmaceutically acceptable salt or derivative thereof, in a hydroalcoholic vehicle comprising water, a short chain alcohol, a monoalkyl ether of diethylene glycol, a pharmaceutically acceptable glycol, and an optional fatty permeation enhancer. These compositions for pharmaceutical delivery may include further components as described herein, for example, the hydroalcoholic vehicle may further comprise a cosolvent(s), a penetration enhancer(s), a buffering agent(s), a preservative(s), an emollient(s), an humectant(s), and/or a gelling agent(s). Such compositions may be formulated in a variety of ways including wherein the hydroalcoholic vehicle is gellified. These compositions may be used, for example, for transmucosal applications including application to nasal, sublingual and buccal tissues.

In a further aspect, the present invention includes methods of manufacturing the compositions described herein for pharmaceutical drug delivery. In one embodiment, the method of manufacturing comprises mixing the components to yield a homogeneous gel, wherein the pH of the gel is between about pH 4.5 and about pH 8.5 (exemplary components include, but are not limited to the following: a therapeutically effective amount of lofexidine, or a pharmaceutically acceptable salt or derivative thereof, a primary vehicle comprising water, at least one short-chain alcohol, a monoalkyl ether of diethylene glycol, a pharmaceutically acceptable glycol, an optional fatty permeation enhancer). These methods may include addition of further components as described herein, for example, the hydroalcoholic vehicle may further comprise cosolvent(s), penetration enhancer(s), buffering agent(s), preservative(s), emollient(s), humectant(s), and/or gelling agent(s). The method provides a gel suitable for transmucosal delivery of lofexidine. Further, a method of manufacturing may further include dispensing the pharmaceutical composition into one or more containers (for example, a unit dose container (e.g., a flexible, foil packet, further comprising a liner) or a multiple dose container).

In another aspect, the present invention includes methods for administering lofexidine to a human subject in need thereof. For example, the method may comprise providing a composition of the present invention for transmucosal pharmaceutical delivery of lofexidine. Doses of the compositions of the present invention may, for example, be a gel applied to the nasal, sublingual or buccal tissues Further, doses of the compositions of the present invention may be applied in a single or in divided doses. In one embodiment, the composition is applied as one or more daily dose of the gel to nasal, sublingual or buccal mucosa of the subject in an amount sufficient for the lofexidine to achieve therapeutic concentration in the bloodstream. Further dosage forms of the compositions of the present invention can be determined by one of ordinary skill in the art in view of the teachings presented herein. The compositions of the present invention may be applied to a mucosal membrane using a variety of means, including, but not limited to a pump-pack, a brush, a swab, a finger, a hand, or other applicator.

The methods of manufacturing of the present invention may include dispensing compositions of the present invention into appropriate containers. The compositions of the present invention may be packaged, for example, in unit dose or multi-dose containers. The container typically defines an inner surface that contains the composition. Any suitable container may be used. The inner surface of the container may further comprise a liner or be treated to protect the container surface and/or to protect the composition from adverse affects that may arise from the composition being in contact with the inner surface of the container. Exemplary liners or coating materials include, but are not limited to high density polyethylene, low density polyethylene, very low density polyethylene, polyethylene copolymers, thermoplastic elastomers, silicon elastomers, polyurethane, polypropylene, polyethylene terephthalate, nylon, flexible polyvinylchloride, natural rubber, synthetic rubber, and combinations thereof. Liners or coating material are typically substantially impermeable to the composition and typically to the individual components of the composition.

A number of types of containers are known in the art, for example, packets with rupturable barriers (see, for example, U.S. Pat. Nos. 3,913,789, 4,759,472, 4,872,556, 4,890,744, 5,131,760, and 6,379,069), single-use packets (see, for example, U.S. Pat. Nos. 6,228,375, and 6,360,916), tortuous path seals (see, for example, U.S. Pat. Nos. 2,707,581, 4,491,245, 5,018,646, and 5,839,609), and various sealing valves (see, for example, U.S. Pat. Nos. 3,184,121, 3,278,085, 3,635,376, 4,328,912, 5,529,224, and 6,244,468). One example of a unit dose container is a flexible, foil packet with a polyethylene liner.

Containers/delivery systems for the compositions of the present invention may also include a multi-dose container providing, for example a fixed or variable metered dose application. Multi-dose containers include, but are not limited to, a metered dose aerosol, a stored-energy metered dose pump, or a manual metered dose pump. In preferred embodiments, the container/delivery system is used to deliver metered doses of the compositions of the present invention for application to the nasal, sublingual and bucccal mucosa of a subject. Metered dose containers may comprise, for example, an actuator nozzle that accurately controls the amount and/or uniformity of the dose applied. The delivery system may be propelled by, for example, a pump pack or by use of propellants (e.g., hydrocarbons, hydro fluorocarbons, nitrogen, nitrous oxide, or carbon dioxide). Preferred propellants include those of the hydrofluorocarbon (e.g., hydrofluoroalkanes) family, which are considered more environmentally friendly than the chlorofluorocarbons. Exemplary hydrofluoroalkanes include, but are not limited to, 1,1,1,2-tetrafluoroethane (HFC-134(a)), 1,1,1,2,3,3,3,-heptafluoropropane (HFC-227), difluoromethane (HFC-32), 1,1,1-trifluoroethane (HFC-143(a)), 1,1,2,2-tetrafluoroethane (HFC-134), 1,1-difluoroethane (HFC-152a), as well as combinations thereof. Particularly preferred are 1,1,1,2-tetrafluoroethane (HFC-134(a)), 1,1,1,2,3,3,3,-heptafluoropropane (HFC-227), and combinations thereof. Many pharmaceutically acceptable propellants have been previously described and may be used in the practice of the present invention in view of the teachings presented herein. The delivery system should provide dose uniformity. In a preferred embodiment, airless packaging with excellent barrier properties is used to prevent degradation of lofexidine, for example, airless metered-dose pumps wherein the composition comprising lofexidine is packaged in collapsible aluminum foils. Accurate dosing from such pumps ensures reproducibility of dose.

The present invention further includes methods for administering a composition of the present invention to a subject in need thereof. Compositions of the present invention comprising lofexidine can be employed, for example, for the treatment of a variety of conditions and/or disease states which have been historically treated by oral doses of lofexidine.

The lofexidine compositions of the present invention may be self-applied by a subject in need of treatment or the composition may be applied by a care-giver or health care professional.

The present invention also provides a method of treating and providing a fast relief from opiates withdrawal symptoms, migraine, neuropathic pain, and other therapeutic indications related to lofexidine, comprising administering to a subject in need thereof a pharmaceutically effective amount of lofexidine through nasal, buccal, and/or sublingual administration.

More particularly, the present invention concerns the transmucosal administration of lofexidine. “Lofexidine” refers to the compound: 2-[1-(2,6 dichlorophenoxy)ethyl]-4,5-dihydro-1H-Imidazole, and has the following formula:

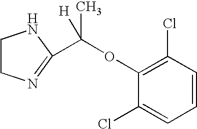

In the present invention, lofexidine can exist in a free base form or as any pharmaceutically acceptable salt. Pharmaceutically acceptable salt, refers to pharmaceutically acceptable salts of lofexidine which are derived from a variety of organic and inorganic counter ions that are well known in the art and include, by way of example only, hydrochloride, hydrobromide, tartrate, mesylate, acetate, maleate, oxalate and the like.

For the purposes of the present invention, lofexidine hydrochloride is preferred; however, other pharmacologically acceptable moieties thereof can be utilized as well.

The term “lofexidine” as used herein includes the free base form of this compound as well as pharmacologically acceptable acid addition salts thereof.

The lofexidine and lofexidine salts for use according to the invention may be in the form of a free amine (i.e. —NH—) or more preferably in the form of a pharmaceutically acceptable salt. In one embodiment, the salts are acid addition salts with physiologically acceptable organic or inorganic acids. Suitable acids include, for example, hydrochloric, hydrobromic, phosphoric, sulphuric and sulphonic acids. In another embodiment, the salts are acid addition salts with hydrochloric acid. Procedures for salt formation are conventional in the art.

In one embodiment, the lofexidine for use in the invention is an enantiomerically pure (e.g. it has an enantiomeric excess of at least 90%, in another embodiment at least 95%, in yet another embodiment at least 99% by weight). In one embodiment, the lofexidine enantiomer for use in the invention is (−)-lofexidine. In another embodiment, the pharmaceutically acceptable salts of lofexidine are those formed from (−)-lofexidine. Enantiomerically pure lofexidine and pharmaceutically acceptable salts thereof may be prepared by conventional procedures described in the art (e.g. as described in J. Med. Chem., 1986, 29, 991183-1188).

The lofexidine therapeutic effect can be achieved to a degree sufficient to cause a relief of opiate addiction symptoms, migraine or treatment of neuropathic pain by the transmucosal administration of lofexidine through nasal, sublingual, and/or buccal delivery so as to maintain an adequate plasma concentration of lofexidine. The amount of lofexidine administered is an amount sufficient to cause therapeutic effect but is low enough not to cause substantial intolerable adverse side effects. As used herein, “substantial intolerable adverse side effects” include those effects caused by either the delivery system or the alpha two receptor agonist which are incompatible with the health of the user or which are so unpleasant as to discourage the continued use of the composition. Such effects include, for example, hypotension, nausea, vomiting, impaired vision, and diaphoresis.

In one embodiment, a “detoxifying amount of lofexidine” includes an effective amount of lofexidine which may substantially saturate, bind to, or block an effective number of the opioid receptors in a subject. The terms “substantially saturate” and “substantially block” an effective number of opioid receptors include about 75%, about 80%, about 85%, about 90%, about 95%, or higher, saturation or blockage of the opioid receptors in a subject.

In one aspect, a detoxifying amount comprises about 0.5 mg to about 10 mg of lofexidine. The dosage of lofexidine may be about 1 mg to about 8 mg, or about 2 mg to about 6 mg, or about 3 mg to about 5 mg, or about 1 mg, 2 mg, 3 mg, 4 mg, 5 mg, 6 mg, 7 mg 8 mg, 9 mg or 10 mg of lofexidine.

The term “opioid” refers to compounds or compositions including metabolites of such compounds or compositions which bind to specific opioid receptors and have agonist (activation) or antagonist (inactivation) effects at these receptors, such as opioid alkaloids, including the agonist morphine and its metabolite morphine-6-glucuronide and the antagonist naltrexone and its metabolite and opioid peptides, including enkephalins, dynorphins and endorphins. The opioid can be present as a member selected from an opioid base and au opioid pharmaceutically acceptable salt. The pharmaceutically acceptable salt embraces an inorganic or an organic salt. Representative salts include hydrobromide, hydrochloride, mucate, succinate, n-oxide, sulfate, malonate, acetate, phosphate dibasic, phosphate monobasic, acetate trihydrate, bi(heptafluorobutyrate), maleate, bi(methylcarbamate), bi(pentafluoropropionate), mesylate, bi(pyridine-3-carboxylate), bi(trifluoroacetate), bitartrate, chlorhydrate, fumarate and sulfate pentahydrate. The term “opiate” refers to drugs derived from opium or related analogs.

Despite the overall popularity of other delivery methods, oral transmucosal delivery through buccal and/or sublingual mucosa, in addition to intranasal delivery system, are particularly advantageous delivery routes. One of the advantages of these delivery systems is that they are non-invasive.

Furthermore, transmucosal delivery generally has better patient compliance, less risk of infection and lower cost than invasive procedures such as injection and implantation. It also has much shorter onset time, i.e., the time from administration to therapeutic effect, than the oral delivery. A drug absorbed via the buccal, nasal, and sublingual mucosa will also avoid first pass metabolism, in which the drug is metabolized in the gastrointestinal tract and liver. Similarly, a drug absorbed via these routes avoids the variability in gastric emptying time commonly observed in patients with proximal gastrointestinal motility syndromes, allowing for greater predictability in obtaining therapeutic blood levels. Such transmucosal delivery systems are simple and dosage forms can be administered by the caregiver or the patient with minimal discomfort.

In one embodiment, the pharmaceutical compositions of the present invention further comprise an effective amount of at least one sedative. In another embodiment, the sedative is selected from the group consisting of antipsychotics, atypical antipsychotics, alpidem, amobarbital, antihistamines, barbiturates, benzodiazepines, chloral hydrate, chlorazepate, chlordiazepoxide, clonazepam, clonidine, diazepam, diethyl ether, dimenhydrinate, diphenhydramine, doxylamine, ethchlorvynol, flunitrazepam, gamma-hydroxybutyrate, glutethimide, herbal sedatives, imidazopyridines, kava, lorazepam, meprobamate, methaqualone, methyl trichloride, methyprylon, olanzapine, phenabarbitol, pentobarbital, promethazine, pyrazolopyrimidines, seroquel, secobarbital, tiagabine, tranquilizers, zaleplon, zolpidem, a pharmaceutically acceptable salt or complex thereof, a combination thereof, and a pharmaceutical composition comprising the same.

The method further comprises administering to the subject an effective amount of at least one opioid before or concurrent with lofexidine treatment.

In one embodiment, the pharmaceutical compositions of the present invention further comprise an effective amount of at least one opioid. In another embodiment, the opioid is selected from the group consisting of opium, morphine, heroin, pethidine, methadone, buprenorphine, butorphanol, codeine, fentanyl, hydrocodone, hydromorphone, levorphanol, meperidine, oxycodone, pentazocine, propoxyphene, or tramadol, pharmaceutical formulations, pharmaceutical salts, or mixtures or combinations there of.

In one embodiment, the pharmaceutical compositions of the present invention further comprise an effective amount of at least one opioid antagonist. In another embodiment, the opioid antagonist is selected from the group consisting of 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof.

In one embodiment, the present invention provides for a method for treating an opiate addiction in a subject comprising administering to a subject with an opiate addiction, an effective amount of a lofexidine transmucosally on a treatment day. The method may further comprise administering to the subject an effective amount of an opiate on at least one treatment day. The method may further comprise administering to the subject an effective amount of a sedative on at least one treatment day. In one embodiment, the number of days of treatment range from about 2 days to about 20 days.

The compositions according to the present invention can be administered, for example, as a spray, drop, suspension, gel, ointment, cream or powder.

The administration of a composition can also include using a pressurized container, strip, or a dropper containing a composition of the present invention.

The composition according to the present invention can be administered, for example, as solid sublingual or buccal transmucosal delivery system such as, but not limited to, a chewing gum, a patch, a lozenge, a tablet, a troche, a pastille, a sachet, and a rapid disintegrating tablet.

Lofexidine can also be brought into a viscous basis via systems conventionally used, for example, natural gums, methylcellulose and derivatives, acrylic polymers (carbopol) and vinyl polymers (polyvinylpyrrolidone).

In the present compositions, many other excipients known in the art can be added such as preservatives, surfactants, co-solvents, adhesives, antioxidants, buffers, viscosity enhancing agents and agents to adjust the pH and the osmolarity.

As set forth previously, the solid transmucosal delivery systems that can be used with the present invention can take forms including, but not limited to, a powder, a chewing gum, a patch, a lozenge, a tablet, a troche, a pastille, a sachet, and a rapid disintegrating tablet.

In one embodiment, the formulation may be presented as capsules, tablets, caplets, pills, powders, granules or a suspension suitable for transmucosal delivery, prepared by conventional means with pharmaceutically acceptable excipients, e.g., with conventional additives or fillers such as lactose, mannitol, corn starch or potato starch; with binders or binding agents such as crystalline cellulose, cellulose derivatives, acacia, corn starch (including pregelatinized) or gelatins; with disintegrators or disintegrants such as corn starch, potato starch or sodium carboxymethyl-cellulose; or with lubricants or wetting agents such as talc or magnesium stearate. Tablets may be coated, including by methods well known in the art. The formulation may be presented as an immediate-release or as a slow-release, sustained-release or controlled-release form. The formulation may also be presented as a solid drug matrix, for example, on a handle. In another embodiment, the formulation may be presented as liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., methyl or propyl-p-hydroxybenzoates or sorbic acid).

For transmucosal administration, the compounds may be combined with penetration enhancers such as propylene glycol, polyethylene glycol, isopropanol, ethanol, oleic acid, N-methylpyrrolidone, or the like, which increase the permeability of the mucosa to the compounds, and permit the compounds to penetrate through the mucosal membranes and into the bloodstream. The compound/enhancer compositions also may be combined additionally with a polymeric substance such as ethylcellulose, hydroxypropyl cellulose, ethylene/vinylacetate, polyvinyl pyrrolidone, or the like, to provide the composition in gel form, which can be dissolved in solvent such as methylene chloride, evaporated to the desired viscosity, and then applied to nasal, sublingual, or buccal mucosa.

The composition may further include additional pharmaceutical ingredients to provide desirable characteristics, such as aesthetically pleasing qualities, improved taste, and the like, to otherwise render the dosage formulation more likely to be administered by the patient. Examples of desirable ingredients include, without limitation, penetration enhancers, colorants, flavorings agents, solvents and co-solvents, coating agents, direct compression excipients, disintegrants, glidants, lubricants, polishing agents, suspending agents, sweetening agents, anti-adherents, binders, and diluents. The ingredients may also include preservatives, emulsifying agents, antioxidants, plasticizers, surfactants, tonicity agents, viscosity increasing agents and combinations thereof. Examples of useful additives include, without limitation, propylene glycol, polyethylene glycol, orange, cherry, mint, and strawberry flavors and other commonly utilized ingredients.

The components of the composition may be formulated in any suitable orally dissolvable dosage form to deliver the lofexidine to the sublingual and buccal oral mucosal tissue. For example, suitable formulations include, without limitation, solid formulations such as a lozenge, a lollipop, a troche, a chewable gum, a solid candy, a granular solid, a chewable tablet or pill, an orally disintegrated tablet or pill, an orally dissolvable tablet, and an orally dissolvable pill.

Such formulations may be prepared utilizing formulating procedures known in this art. For example, there are several ways to create a solid, orally dissolvable formulation, including, but not limited to, wet granulation, co-melt, spray-drying, freeze-drying, and the like. Particularly, solid formulations such as tablets, lozenges and the like may be prepared utilizing such techniques, including wet granulation, co-melt, spray-drying, freeze-drying, and the like.

The process of wet granulation can be outlined as several steps: weighing and blending the ingredients of the composition in the presence of solvent(s), drying the mixture into solid, and milling the solid to proper size.

In the weighing and blending step of wet granulation, proper amounts of the oral dissolution agent(s), lofexidine, and solvent(s) are thoroughly mixed. Additional ingredients may be added to facilitate the mixing of the ingredients. The solvent(s) utilized should dissolve both the lofexidine and oral dissolution agent(s). The end result of this step is a finely blended mixture in which lofexidine and the dissolution agent are mixed at the molecular level. The mixture is then dried and generally ground to a powder so that it can be compressed into solid units. There are several ways to dry the wet granulation mixture depending on the mixture, the solvent, and the equipment. Milling and screening steps are usually used to ensure the proper particle size distribution for compression.

Thus, upon dissolution in the oral cavity, the lofexidine is released and delivered to the sublingual or buccal oral mucosal tissue, resulting in an effective rate of absorption.

As set forth previously, the liquid transmucosal delivery systems that can be used with the present invention can take various forms including, but not limited to, aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

In the present invention, the pH of the compositions could be maintained from about 2.0 to about 10.0. Compositions having a pH of less than about 2.0 or greater than about 10.0 can increase the risk of irritating the mucosal membranes in the nasal, sublingual, and buccal region of a recipient. Further, it is preferable that the pH of the compositions be maintained from about 3.0 to about 7.0. To extend shelf life, preservatives can be added to the present compositions. Suitable preservatives that can be used with the present compositions include benzyl alcohol, parabens, thimerosal, chlorobutanol and benzalkonium chloride and preferably benzalkonium chloride is used. Typically, the preservative will be present in a composition in a concentration of up to about 2% by weight. The exact concentration of the preservative, however, will vary depending upon the intended use and can be easily ascertained by one skilled in the art.

The present invention provides for the compositions as described above which are administered through nasal, sublingual or buccal mucosal membranes to a mammal to treat neuropathic pain, migraine, opiate withdrawal symptoms, and other therapeutic indications related to lofexidine.

The term “subject in need thereof” refers to any animal in need of relief from the symptoms of opiate addiction withdrawal, migraine, neuropathic pain, or conditions that can be treated with lofexidine. Preferably, the subject is a mammal. More preferably, the subject is human.

This invention also includes pharmaceutical compositions, which contain as the active ingredient, one or more of the compounds of the subject invention above, associated with one or more pharmaceutically acceptable carriers or excipients. The excipient employed is typically one suitable for administration to human subjects or other mammals. In making the compositions of this invention, the active ingredient is usually mixed with an excipient, diluted by an excipient. When the excipient serves as a diluent, it can be a solid, semi-solid, or liquid material, which acts as a vehicle, carrier or medium for the active ingredient.

The compositions of the invention can be formulated so as to provide fast and/or sustained release of the active ingredient after administration to the patient by employing procedures known in the art.

The following examples are offered to illustrate this invention and are not to be construed in any way as limiting the scope of this invention.

### EXAMPLES

The following Examples are provided to illustrate certain aspects of the present invention and to aid those of skill in the art in practicing the invention. These Examples are in no way to be considered to limit the scope of the invention in any manner.

### Example 1

### Transmucosal Tablet Formulation

In one embodiment, the invention provides a 0.1 mg strength lofexidine sublingual/buccal tablet having a total tablet weight of about 100 mg, wherein the tablet comprises drug, an absorbent/adsorbent particulate carrier, such as silica; a diluent, such as mannitol; a disintegrant, such as sodium starch glycolate; and a lubricant, such as sodium stearyl fumarate, to facilitate tableting. In such an embodiment, lofexidine is dissolved in polyethylene glycol 400 (PEG 400). The resulted lofexidine mixture is added to the rest of ingredient and processed into a powder mix suitable for use in direct compression tableting. An exemplary formulation in accordance with the described formulation of this embodiment is provided in Table 1, below:

TABLE 1
Lofexidine Sublingual/Buccal Tablet Formulation

| | Amount |
|---|---|
| Ingredient | (mg/tablet) |
| Lofexidine | 0.10 |
| hydrochloride | |
| PEG 400 | 5.90 |
| Sodium Stearyl | 0.50 |
| Fumarate | |
| Sodium Starch | 4.00 |
| Glycolate | |
| Mannitol | 85.00 |
| Silica | 4.50 |

### Example 2

### Transmucosal Spray Solution

The composition comprises lofexidine hydrochloride: 200.0 mg; phosphate buffer (0.05 M, pH 4.4): 200.0 mL and the lofexidine is dissolved in the phosphate buffer and pH of the solution is readjusted to 4.4 if necessary. The solution is placed in an administrator designed to deliver 100 μL of spray for each application. One spray will deliver a total of 0.1 mg of lofexidine hydrochloride.

### Example 3

### Transmucosal Gel Formulation

The composition comprises lofexidine hydrochloride: 1.0 g; Methocel: 3.0 g; Acetate buffer (0.05 M, pH 4-6) 100.0 g; Approximately 70 mL of the acetate buffer is heated to 80° C., and the methocel is dispersed in it with stirring. The lofexidine hydrochloride is dissolved in 30 mL of the acetate buffer at 80° C., and the solution is mixed with the methocel dispersion. The resultant mixture is allowed to stand at room temperature for 3 hours. The gel is placed in an ointment tube equipped with a fine orifice and is applied transmucosally with a finger, a dropper, or cotton tipped applicator. Furthermore, the gel can be casted over a slap and evaporated to produce films suitable for transmucosal delivery.

### Example 4

### An In Vivo Absorption of Lofexidine from the Sublingual Cavity

Experimental Technique

These experiments determine the bioavailability of lofexidine after transmucosal administration to the sublingual tissue in rabbit and compare it to that after intravenous administration.

The transmucosal absorption of lofexidine was measured using an in vivo technique in rabbits. Following introduction of lidocaine local anesthesia, a catheter was placed in the marginal ear artery of the rabbit for blood sample collection. For intravenous administration, a catheter was placed in the marginal ear vein of the rabbit and lofexidine hydrochloride aqueous solution (0.1 mL) was administered; a sterile drug solution was prepared by filtration (double 0.22 μm filters). A dose of 1 mg/kg lofexidine was injected into the marginal ear vein cannula followed by a 0.1 mL flush with 10% (v/v) heparin/normal saline solution to keep the cannula patent.

For sublingual administration, the lofexidine dose (1 mg/kg, 0.1 mL) of the lofexidine formulation was applied to the sublingual mucosa of the rabbit.

Aliquot parts of 1 mL blood samples were collected at predetermined time points, collected into pre-heparinized tubes and immediately placed on ice. Plasma was separated by centrifugation at 3000 rpm for 10 min, placed in polypropylene tubes, and frozen at −20° C. until the time of analysis.

Bioavailability of sublingually administered drug was calculated by comparing the plasma drug concentration between sublingual and intravenous delivery routes and expressed as a percentage of the intravenous bioavailability.

Formulation of the Transmucosal Dosage Form

The formulation instilled into the sublingual area of the rabbits in this experiment had the following composition:
Lofexidine hydrochloride 6000 mg
Isopropanol 2 mL
Phosphate buffer (0.05 M, pH 6)

The lofexidine is dissolved in the isopropanol and the phosphate buffer to make the volume up to 200 mL.

Sample Preparation

The internal standard (IS) solution was prepared by dissolving d⁴-lofexidine in a methanol:water (50:50) mixture to get a (1000 ng/mL) concentration. Plasma samples (50 μL) were spiked with IS solution (10 μL), followed by vortex mixing for 1 minute. The mixture was mixed with acetonitrile (50 μL) to precipitate plasma proteins and allowed to vortex for another minute. After centrifugation for 10 minute at 10,000 rpm, the supernatant was collected into UPLC vials in preparation for analysis by Ultra Performance Liquid Chromatography Mass Spectroscopy (UPLC/MS) system.

Assay Method for Lofexidine

All analytical procedures were performed using a Waters Acquity® Ultra UPLC/MS. An Acquity UPLC BEH Shield RP18 (2.1×100 mm) column (Waters) was used to separate the chemical components at 40° C. The flow rate was 0.3 mL/min of two mobile phases, (A) made of 5 mM ammonium acetate (pH 4) and acetonitrile (90:10) and (B) of acetonitrile, 5 mM ammonium acetate (pH 4) (90:10). The mass spectrometer was operated in the positive electrospray ionization (ESI) mode. The capillary voltage and cone voltage were maintained at 0.6 kV and 35 V, respectively. The source temperature and desolvation temperature were set at 100 and 350° C., respectively. Nitrogen was used as both the cone gas (50 L/h) and the desolvation gas (700 L/h). Mass chromatograms and mass spectral data were acquired and processed by MassLynx software (Waters).

Results

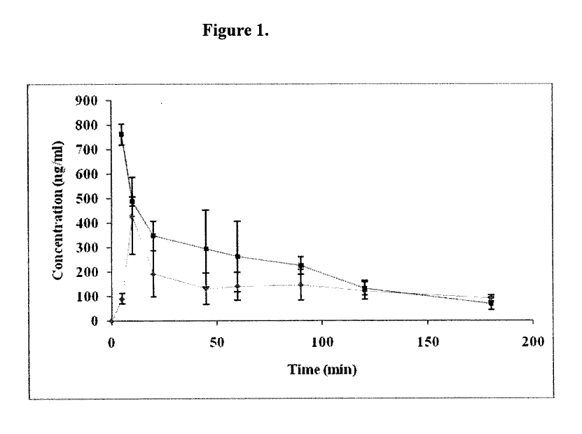

The pharmacokinetic profile of lofexidine was assessed in intact animals over 180 minutes. Mean lofexidine plasma concentration profiles versus time relationship that resulted after sublingual dosing to rabbits in comparison with intravenous administration were illustrated in FIG. 1.

The pharmacokinetic parameters for the sublingual transmucosal dosing route are presented in Table 2.

TABLE 2
Pharmacokinetic parameters following sublingual administration
of lofexidine to rabbits (n = 3)

| Parameter | |
|---|---|
| average and | |
| Unit | Sublingual |
| Cₘₐₓ (ng/mL) | 392 ± 127 |
| tₘₐₓ (min) | 10 |
| t_{1/2} (min) | 75 ± 2.7 |

Comparing the areas under the plasma concentration versus time curves (see FIG. 1) shows that lofexidine is rapidly and completely absorbed following transmucosal administration through the sublingual route, and the peak plasma concentration occurs at approximately 10 minutes following transmucosal administration. These results indicate that the transmucosal route of administration produces a rapid response with great bioavailability around 80% of the administered dose.

While this invention has been described as having a preferred embodiment, it is understood that the invention is not limited to the illustrated and described features. To the contrary, the invention is capable of further modifications, uses, and/or adaptations following the general principles of the invention and therefore includes such departures from the present disclosure as come within the known or customary practice in the art to which the invention pertains, and as may be applied to the central features set forth above, and which fall within the scope of the appended claims.

It would be obvious to those skilled in the art that modifications or variations may be made to the preferred embodiment described herein without departing from the novel teachings of the present invention. All such modifications and variations are intended to be incorporated herein and within the scope of the claims.

What is claimed is:

1. A method for rapidly and reliably delivering lofexidine to the systemic circulation of a patient comprising transmucosal administration of a therapeutically effective amount of lofexidine or a pharmaceutically acceptable free base, salt, ester, amide or prodrug thereof, to a patient in need of such treatment.

2. The method of claim 1 wherein the pharmaceutical composition further comprises a carrier suitable for transmucosal drug administration and, optionally, a permeation enhancer.

3. The method of claim 1 wherein the pharmaceutical composition is administered transmucosally through a delivery route selected from the group consisting of nasal, sublingual, transbuccal and transrectal routes of delivery.

4. The method of claim 1 wherein the dosage form is selected from the group consisting of spray, drops, gels, tablets, troches, lozenges, chewing gum, and patches.

5. The method according to claim 1, wherein lofexidine is administered once-per-day in an amount of between about 0.1 mg and about 5 mg.

6. The method of claim 1, wherein the transmucosal formulation is a tablet.

7. The method of claim 1, wherein the transmucosal formulation is a gel.

8. The method of claim 1, wherein the transmucosal formulation is an adhesive film.

9. The method of claim 2, wherein the pharmaceutical composition further comprises a permeation enhancer selected from the group consisting of fatty acids, fatty acid esters, fatty alcohols, fatty acid esters of lactic acid or glycolic acid, glycerol triesters, glycerol diesters, glycerol monoesters, triacetin, short chain alcohols, and mixtures thereof.

10. The method according to claim 2, wherein the lofexidine is in a formulation that further comprises, by weight of the formulation, an alkanol in an amount between about 5 to 80%, a polyalcohol in an amount between about 1% to 30%, and a permeation enhancer in an amount between about 1 to 30%.

11. The method according to claim 2, wherein the formulation further comprises at least one of a gelling agent, neutralizing agent, buffering agent, moisturizing agent, humectant, surfactant, antioxidant, emollient, or buffer.

12. The method according to claim 11, wherein the formulation is provided in the form of a gel, lotion, cream, ointment, emulsion, or suspension.

13. The method according to claim 2, which further comprises accurately controlling the administration of lofexidine by dispensing the formulation from a metered dosage device.

14. The method according to claim 13, wherein the metered dosage device dispenses a precise amount of lofexidine for self administration upon a transmucosal surface of the subject.

15. A method for administering a transmucosal gel formulation comprising a lofexidine, an alkanol in an amount of about 5 to 80% by weight, a polyalcohol in an amount of about 1% to 30% by weight, and a permeation enhancer in an amount of about 1 to 30% by weight of the formulation.

16. A pharmaceutical composition comprising lofexidine or a pharmaceutically acceptable free base, salt, ester, amide or prodrug thereof and a pharmaceutically acceptable carrier, wherein the composition is provided in a form suitable for transmucosal administration.

17. The pharmaceutical composition of claim 16 wherein the pharmaceutically acceptable carrier is selected from the group consisting of aqueous solution, non-aqueous solution, or a combination of an aqueous solution and a non-aqueous solution thereof

18. The pharmaceutical composition of claim 16 wherein the pharmaceutically accepted carrier further comprises solutions, gels, suspensions, liposomal dispersions, emulsions, microemulsions, nanoparticles and combinations thereof.

19. The pharmaceutical composition of claim 16 wherein the pharmaceutically acceptable carrier is a powder formulation

20. The pharmaceutical composition of claim 19 wherein the pharmaceutically accepted carrier is a powder formulation selected from the group consisting of a simple powder mixtures, powder microspheres, coated powder microspheres, liposomal dispersions and combinations thereof.

21. The pharmaceutical composition of claim 20 wherein the composition is provided in a sublingual or buccal transmucosal solid dosage form.

22. The pharmaceutical composition of claim 21 wherein the transmucosal dosage form is chosen from: a tablet, a chewing gum, a patch, a lozenge, a troche, a pastille, a sachet, and a rapid disintegrating tablet.

23. The pharmaceutical composition of claim 16 wherein the composition comprises lofexidine or a pharmaceutically acceptable salt in a dose from about 0.1 mg to about 5 mg of lofexidine.

24. The pharmaceutical composition of claim 16 wherein the concentration of lofexidine is from about 0.01% to about 90% of the dry matter weight of the composition.

25. The pharmaceutical composition of claim 16 wherein the composition further comprises at least one flavors agent, artificial coloring, sweetener, lubricating agent, disintegration agent, permeation enhancer, lubricating agent, diluent, base, or buffering agent.

26. The pharmaceutical composition of claim 16 wherein the carrier is a hydrolyzable polymer.

27. The pharmaceutical composition of claim 16 wherein the lofexidine is enantiomerically pure.

28. The pharmaceutical composition of claim 16 wherein the lofexidine is (−)-lofexidine.

29. The pharmaceutical composition of claim 16 wherein the lofexidine is lofexidine hydrochloride.

30. The pharmaceutical composition of claim 16, further comprising an additional active agent.

31. The pharmaceutical composition of claim 30 wherein the additional active agent is an opioid analgesic.

32. The pharmaceutical composition of claim 30, wherein the additional active agent is an opioid antagonist.

33. The pharmaceutical composition of claim 32, wherein the opioid antagonist comprises 7-benzylidenenaltrexone, beta-funaltrexamine, buprenorphine, butorphanol, chlornaltrexamine, clocinnamox, connective tissue-activating peptide, cyclazocine, diprenorphine, ICI 154129, levallorphan, meptazinol, methylnaltrexone, N,N-diallyl-tyrosyl-alpha-aminoisobutyric acid-phenylalanyl-leucine, nalbuphine, nalmefene, nalorphine, naloxone, naltrexone, or naltrindole, or mixtures or combinations thereof.

34. A method for the treatment of opiate withdrawal symptoms, migraine, neuropathic pain and other therapeutic indications related to lofexidine, by transmucosal administration of a therapeutically effective amount of the pharmaceutical composition of claim 16 to the subject in need of such treatment.

35. The method of claim 34 wherein the pharmaceutical composition is administered transmucosally through nasal, sublingual or buccal routes of delivery.

36. A system for dispensing a precise amount of a fluid medicament, the system comprising a storage unit for retaining the medicament containing an active agent, and a dispenser unit for releasing a predetermined amount of the medicament upon activation by a user such that the predetermined amount of the active agent is released when desired, wherein the medicament is lofexidine.

37. The system according to claim 36, wherein the dispenser unit comprises a pressure-operable pump, which dispenses the predetermined amount of the medicament upon activation.
